(19) United States
(12) Patent Application Publication
(10) Pub. No.: US 2022/0339500 A1
(43) Pub. Date: Oct. 27, 2022

(54) INFORMATION GENERATION DEVICE, INFORMATION GENERATION METHOD, AND RECORDING MEDIUM

(71) Applicant: NEC Corporation, Minato-ku, Tokyo (JP)

(72) Inventors: Tomohiro MIWA, Tokyo (JP); Yoshikazu ARAI, Tokyo (JP)

(73) Assignee: NEC Corporation, Minato-ku, Tokyo (JP)

(21) Appl. No.: 17/641,193

(22) PCT Filed: Sep. 17, 2020
(86) PCT No.: PCT/JP2020/035302

§ 371 Date: Mar. 8, 2022

(30) Foreign Application Priority Data
Sep. 20, 2019 (JP) 2019-171531

(51) Int. Cl. A63B 24/00; G06V 40/20; G16H 20/30

(52) U.S. Cl. CPC A63B 24/0075; G06V 40/23; A63B 24/0062; G16H 20/30; A63B 2220/05; A63B 2220/806

(57) ABSTRACT

The information generation device stores a model that is generated based on a user information relating to a user, a motion information relating to a motion of each part of a body of the user, and an evaluation report including a comment and an exercise program created by a specialist. The information generation device creates an exercise program for each part of one user, from the user information of the one user and the motion information of each part of the body of the one user based on the model, and also creates a comment indicating a basis of creating the exercise program for each part for the one user. Then, the information generation device presents an evaluation report including the exercise program for each part and the comment indicating the basis.

### TECHNICAL FIELD

The present disclosure relates to a device for proposing an exercise program suitable for a user.

### BACKGROUND ART

In the fields of medical care, nursing, healthcare and the like, many users exercise for the purpose of improving and strengthening of physical function, preventing nursing, or the like. At this time, specialists such as physiotherapists and trainers diagnose users directly and provide exercise programs corresponding to individual conditions.

The number of specialists, such as physiotherapists, is small relative to the number of facilities and users. In order to diagnose each user directly and provide an exercise program, the specialists need to take charge of multiple facilities and provide exercise programs for multiple users. Therefore, the costs required, such as the movement between facilities and the time required for preparation of documents, are large.

For this reason, there has been proposed a device which creates an exercise menu based on the information such as the condition of the user. For example, Patent Document 1 discloses an exercise menu proposal system which detects the walking ability including the symmetry of the stride length of the left and right feet from the walking motion of the subject, determines the falling risk from the walking ability, and proposes an exercise menu according to the walking ability or the falling risk. Patent Document 2 discloses a cognitive function evaluation assisting device for evaluating the degree of cognitive function of a person to be measured by checking the walking parameter calculated from the walking locus of the person to be measured, based on the relationship between the walking parameter of a general person and the degree of cognitive function.

### PRECEDING TECHNICAL DOCUMENTS

### Patent Documents

Patent Document 1: Japanese Patent Application Laid-Open under No. 2009-261595
Patent Document 2: Japanese Patent Application Laid-Open under No. 2018-161258

### SUMMARY

### Problem to be Solved

While the above patent documents propose and evaluate exercise menus related to the user's walking, many users also wish to create exercise programs for intensively enhancing and rehabilitating specific parts of the body.

It is an object of the present disclosure to provide an information generation device capable of creating an appropriate exercise program for each part of a user's body.

### Means for Solving the Problem

According to one aspect of the present disclosure, there is provided an information generation device comprising:

an acquisition means configured to acquire a user information relating to a user, a motion information relating to a motion of each part of a body of the user, and an evaluation report including a comment and an exercise program created by a specialist;

a storage means configured to store a model generated based on a plurality of the user information, a plurality of the motion information, and a plurality of the evaluation report;

a first creating means configured to create an exercise program for each part of one user, from the user information of the one user and the motion information of each part of the body of the one user based on the model;

a second creating means configured to create a comment indicating a basis of creating the exercise program for each part for the one user; and

a generation means configured to generate an evaluation report including the exercise program for each part and the comment indicating the basis.

According to another aspect of the present disclosure, there is provided an information generation method comprising:

acquiring a user information relating to a user, a motion information relating to a motion of each part of a body of the user, and an evaluation report including a comment and an exercise program created by a specialist;

acquiring a model generated based on a plurality of the user information, a plurality of the motion information, and a plurality of the evaluation report;

creating an exercise program for each part of one user, from the user information of the one user and the motion information of each part of the body of the one user based on the model;

creating a comment indicating a basis of creating the exercise program for each part for the one user; and

generating an evaluation report including the exercise program for each part and the comment indicating the basis.

According to still another aspect of the present disclosure, there is provided a recording medium recording a program causing a computer to execute a process of:

acquiring a user information relating to a user, a motion information relating to a motion of each part of a body of the user, and an evaluation report including a comment and an exercise program created by a specialist;

acquiring a model generated based on a plurality of the user information, a plurality of the motion information, and a plurality of the evaluation report;

creating an exercise program for each part of one user, from the user information of the one user and the motion information of each part of the body of the one user based on the model;

creating a comment indicating a basis of creating the exercise program for each part for the one user; and

generating an evaluation report including the exercise program for each part and the comment indicating the basis.

### Effect

According to the present invention, it is possible to provide an information generation device capable of creating an appropriate exercise program for each part of a user's body.

### BRIEF DESCRIPTION OF THE DRAWINGS

FIG. 1. shows a schematic configuration of an information generation device according to a first example embodiment.

FIG. 2 is a block diagram showing a hardware configuration of an evaluation report creating device.

FIG. 3 is a block diagram showing a functional configuration of the evaluation report creating device.

FIG. 4 is an example of user information stored in a user information database.

FIG. 5 is an example of an evaluation report.

FIG. 6 is an example of comments described in a comment section of the evaluation report.

FIG. 7 is an example of a matrix of exercise programs.

FIG. 8 shows examples of explanatory variables for an AI model.

FIG. 9 is a flowchart of an evaluation report creating process.

FIG. 10 is a block diagram showing a functional configuration of an information generation device according to a second example embodiment.

### EXAMPLE EMBODIMENTS

Hereinafter, example embodiments of the present disclosure will be described with reference to the accompanying drawings.

### First Example Embodiment

First, a first example embodiment of the present disclosure will be described.

(Overall Configuration)

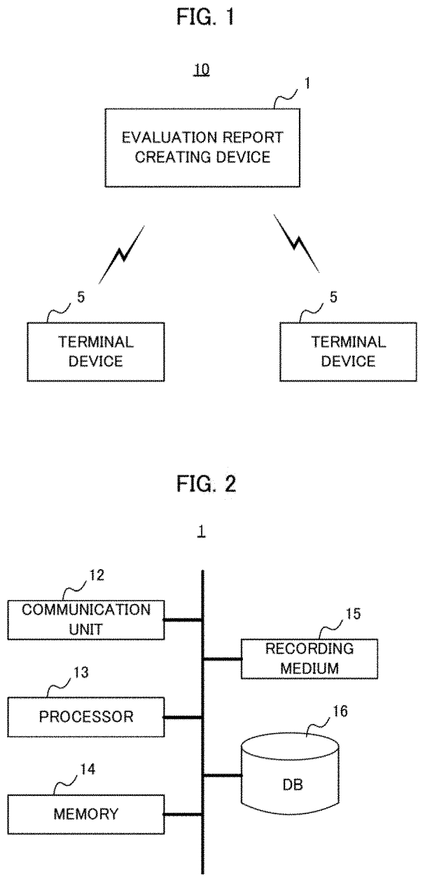

FIG. 1 shows a schematic configuration of an information generation device according to the first example embodiment. The information generation device 10 may be used in the scene of providing an exercise program (for rehabilitation or health promotion) to a user such as a patient or a resident in a hospital, a nursing care facility, or a fitness gym. As shown, the information generation device 10 includes an evaluation report creating device 1 and terminal devices 5. The evaluation report creating device 1 is constituted by a server device, for example, and provided in a hospital, a nursing care facility, a fitness gym, or the like (hereinafter collectively referred to as “facility”) where the information generation device 10 is installed. The evaluation report creating device 1 creates an appropriate exercise program for each part of the user's body, and presents the evaluation report to the user, together with the evidence indicating that the exercise program is appropriate.

The terminal device 5 is a personal computer (PC), a tablet terminal, a smart phone, or the like. As an example, the terminal device 5 is intended to be carried by the user who uses the information generation device 10. In this case, the user brings his/her own tablet terminal or smartphone, and uses it in the facility. On the other hand, when the PC of the user's home is used as the terminal device 5, the PC may be connected to the evaluation report creating device 1 of the facility via a communication line. As another example, a PC or a tablet terminal shared by a plurality of users may be installed in the facility as the terminal device 5.

(Hardware Configuration)

FIG. 2 is a block diagram illustrating a hardware configuration of the evaluation report creating device 1. As illustrated, the evaluation report creating device 1 includes a communication unit 12, a processor 13, a memory 14, a recording medium 15, and a database (DB) 16.

The communication unit 12 communicates with a plurality of terminal devices 5 by a wired or wireless connection. Specifically, the communication unit 12 is used for transmitting the user information or the video of the exercising user from the terminal device 5 to the evaluation report creating device 1 and for transmitting the evaluation report from the evaluation report creating device 1 to the terminal device 5.

The processor 13 is a computer such as a CPU (Central Processing Unit) or a CPU with a GPU (Graphics Processing Unit), and controls the entire evaluation report creating device 1 by executing a program prepared in advance. The memory 14 is composed of a ROM (Read Only Memory), a RAM (Random Access Memory), or the like. The memory 14 stores various programs to be executed by the processor 13. The memory 14 is also used as a work memory during the execution of various processes by the processor 13.

The recording medium 15 is a non-volatile and non-transitory recording medium such as a disk-shaped recording medium or a semiconductor memory, and is configured to be detachable from the evaluation report creating device 1. The recording medium 15 records various programs to be executed by the processor 13. When the evaluation report creating device 1 executes various kinds of processes, the programs recorded on the recording medium 15 are loaded into the memory 14 and executed by the processor 13.

The DB 16 stores various types of data used in the evaluation report creating device 1. Specifically, the DB 16 includes a video DB, a user information DB, a report DB, and the like, which will be described later. In addition to the above, the evaluation report creating device 1 may include an input device such as a keyboard and a mouse, or a display device.

(Functional Configuration)

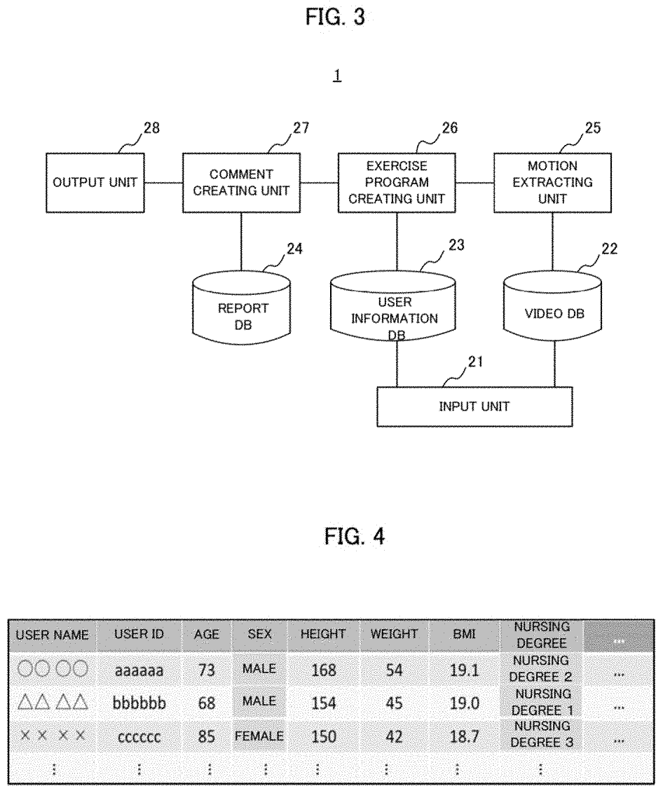

FIG. 3 is a block diagram illustrating a functional configuration of the evaluation report creating device 1. As illustrated, the evaluation report creating device 1 includes an input unit 21, a video DB 22, a user information DB 23, a report DB 24, a motion extracting unit 25, an exercise program creating unit 26, a comment creating unit 27, and an output unit 28.

The input unit 21 is constituted by the communication unit 12 described above, and receives the user information and the exercise video of the user from the terminal device 5. The input unit 21 stores the received exercise video in the video DB 22 and stores the received user information in the user information DB 23.

The video DB 22 stores the exercise video of the user received from the terminal device 5. The “exercise video” is a video showing a user performing a certain exercise motion, and is typically taken by a video-capturing function of the terminal device 5. An example of the exercise video is a video showing a state of performing a TUG (Timed UP and Go: Time Up and Go) test. This test is used for the evaluation of the walking ability of the aged people. However, in the present example embodiment, the exercise motion performed in the exercise video is not limited to the TUG test.

The user information DB 23 stores the user information about each user received from the terminal device 5. FIG. 4 shows an example of user information stored in the user information DB 23. In this example, the user information includes information for health management such as “age”, “sex”, “height”, “weight”, “BMI”, and “nursing degree” in addition to “user name” and “user ID” of the user.

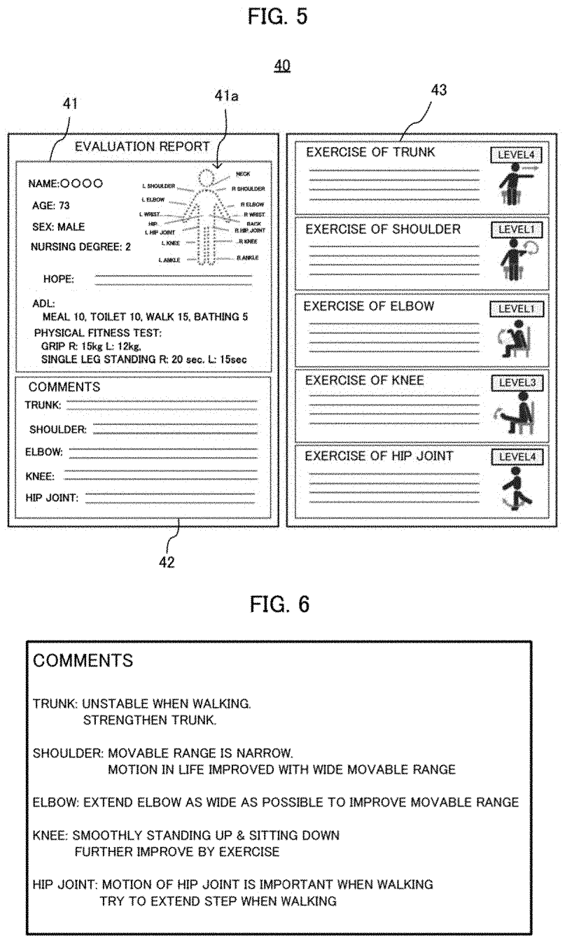

The report DB 24 stores a large number of evaluation reports that specialists have actually created based on the user information and the exercise video in the past. FIG. 5 shows an example of the evaluation report. As shown, the evaluation report 40 includes a user information section 41, a comment section 42, and an exercise program section 43. The user information section 41 describes the user information registered for the user. This user information is stored in the user information DB 23. Further, the user information section 41 is also provided with an area 41a illustrating the condition of each part of the user's body, and an area describing the user's hope, the scoring result of the activity of daily living (ADL), the result of the physical fitness test, and the like.

In the comment section 42, comments prepared for each part of the user's body are described. FIG. 6 shows an example of the comments described in the comment section 42. Each comment includes the current condition of each part of the user's body, and the exercise proposed to improve the condition. These comments were made by specialists based on the user information and the exercise videos of user. The specialist checks the user information and the exercise video, and describes improvement points and attention in the comment section for each part of the body set beforehand from the condition of the user.

In the exercise program section 43, the exercise program proposed to the user is described separately for each part of the body. The exercise program for each part is associated with the comment for each part of the body described in comment section 42. That is, for each part of the body, the exercise program created by the specialist based on the condition and the improvement points described in the comment section 42 is described in the exercise program section 43. Therefore, the comment for each part of the body described in the comment section 42 serves as a basis indicating why the exercise program shown in the exercise program section 43 was selected, i.e., indicating that the exercise program is suitable for that user. Therefore, by referring to the comment section 42 and the exercise program section 43, the user can do the proposed exercise program while being conscious of the condition and the improvement points of each part of its own body.

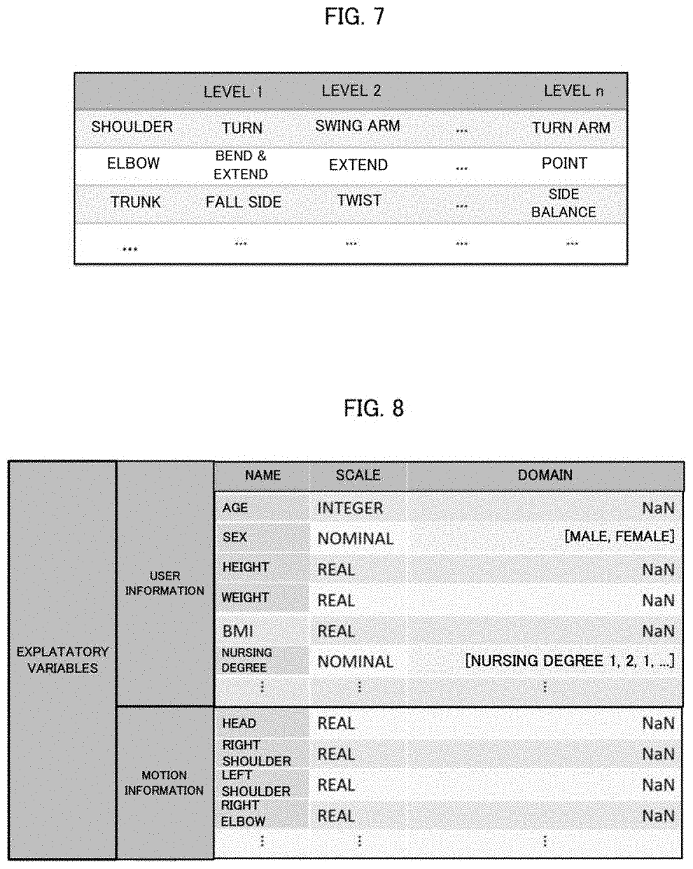

Further, in the exercise program section 43, the exercise program proposed for each part of the body is shown together with the difficulty level. By this, the user is motivated to promote the health and is able to promote the health. Regarding the difficulty of the exercise program, a matrix is prepared in which a plurality of exercises with different degrees of difficulty are defined for each part of a body. FIG. 7 shows an example of the matrix of the exercise programs. By referring to this matrix, the specialist selects the exercise of the difficulty level suitable for the comment content in the comment section 42 for each part of the user's body, and creates the exercise program. The exercise program for each part of the body thus created is shown in the exercise program section 43.

The motion extracting unit 25 extracts the motion information from the exercise video. Specifically, the motion extracting unit 25 extracts the state of the motion of each part of the body from the exercise video, generates motion information such as the movable range of each part and the speed of the exercise, and outputs it to the exercise program creating unit 26.

The exercise program creating unit 26 creates an exercise program to be proposed to the user based on the user information obtained from the user information DB 23 and the motion information obtained from the motion extracting unit 25. The exercise program creating unit 26 includes an AI (Artificial Intelligence) model. This AI model is generated by heterogeneous mixed learning in which each item and motion information of user information are used as the explanatory variables as illustrated in FIG. 8 and each exercise on the matrix of the exercise programs is used as an objective variable. In the model generated by the heterogeneous mixed learning, the cases are divided by the tree structure, and the prediction is carried out using the prediction formula combining the different explanatory variables in each case. It should be noted that the model is preferably relearned periodically or every time a predetermined amount of new user information or exercise video is registered.

Specifically, when the user information and the motion information are inputted, the exercise program creating unit 26 determines one prediction formula for each part of the body set in the matrix of the exercise program according to the branching condition of the tree structure, and selects the exercise program for each part of the body by the prediction formula. At this time, information on the explanatory variables contributing to each selected exercise can be obtained from the explanatory variables used for the branching conditions and the explanatory variables used for the prediction formulas. The information such as the explanatory variables used in the creation of the exercise program and the exercise program for each part of the body thus created are sent to the comment creating unit 27.

The comment creating unit 27 creates the comment for each part of the body to create an evaluation report. First, the comment creating unit 27 searches the user information DB 23 for the past user information most similar to the newly registered user information for each exercise, with respect to the values of the explanatory variables obtained from each exercise indicated by the created exercise program. Then, the comment creating unit 27 extracts the comment about the corresponding body part described in the evaluation report created from the respective past user information, and uses them as the comment about the corresponding body part of the new evaluation report.

Further, the comment creating unit 27 creates a new evaluation report as illustrated in FIG. 5 using the user information acquired from the user information DB 23, the comment for each part of the body created as described above, and the exercise program for each part of the body created by the exercise program creating unit 26, and supplies the new evaluation report to the output unit 28.

The output unit 28 is constituted by the communication unit 12 shown in FIG. 2, and transmits the created new evaluation report to the terminal device 5. The user can confirm the transmitted evaluation report and do the proposed exercise program.

(Evaluation Report Creating Process)

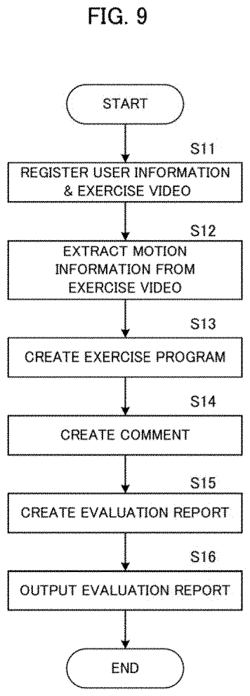

Next, an evaluation report creating process by the evaluation report creating device 1 will be described. FIG. 9 is a flowchart of the evaluation report creating process. This process is implemented by the processor 13 shown in FIG. 2 executing a program prepared in advance.

First, through the terminal device 5, the user information and the exercise video of the user subjected to the creation of the report are registered in the user information DB 23 and the video DB 22, respectively (step S11). Next, the motion extracting unit 25 extracts the motion information from the registered exercise video (step S12). Next, the exercise program creating unit 26 creates the exercise program for each part of the body based on the user information acquired from the user information DB 23 and the motion information acquired from the motion extracting unit 25 (step S13).

Next, the comment creating unit 27 creates the comment for each part of the user's body using the information such as the exercise program acquired from the exercise program creating unit 26 and the previous evaluation report stored in the report DB 24 (step S14). Then, the comment creating unit 27 creates the evaluation report including the created comment, the user information, and the exercise program (step S15). The output unit 28 outputs the created evaluation report to the terminal device 5 of the user (step S16). In this way, the evaluation report creating process is completed.

(Effects)

As described above, according to the information generation device 10 of the present example embodiment, even a person (a nurse, a caregiver, a person without sufficient trainer experience, or the like) who is not a specialist can automatically create an exercise program according to the condition of an individual user (health condition, presence or absence of injury, degree or severity of injury, illness, degree of nursing, physical condition, physical ability, etc.). Particularly, according to the present example embodiment, it is possible to create an exercise program for each part of the user's body according to the condition of each user. Therefore, it is possible to create an exercise program for individual users at lower cost compared with the case where a specialist makes an exercise program by directly diagnosing each user. Also, it is possible to make an exercise program more suitable for each user, because the exercise program is created based on the accumulated data of many users.

### Second Example Embodiment

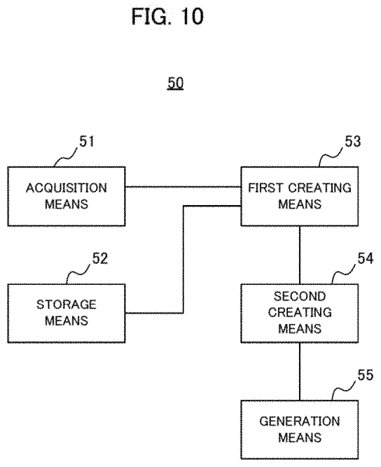

Next, a second example embodiment of the present disclosure will be described. FIG. 10 shows the functional configuration of the information generation device 50 according to the second example embodiment. The hardware configuration of the information generation device 50 according to the second example embodiment is the same as that of the evaluation report creating device 1 shown in FIG. 2.

The information generation device 50 includes an acquisition means 51, a storage means 52, a first creating means 53, a second creating means 54, and a generation means 55. The acquisition means 51 acquires a user information about the user, a motion information about a motion of each part of the body of the user, and an evaluation report including a comment and an exercise program created by a specialist. The storage unit 52 stores a model generated based on a plurality of user information, a plurality of motion information, and a plurality of evaluation reports.

The first creating means 53 create an exercise program for each part of one user based on the above-described model, from the user information of one user and the motion information of each part of the body of the one user. The second creating means 54 creates a comment indicating a basis of creating the exercise program for each part of the body of the one user. Then, the generation means 55 generates an evaluation report including the exercise program for each part of the body and the comment indicating the basis.

According to the second example embodiment, it is possible to create an appropriate exercise program for each part of the user's body.

[Modification]

(Modification 1) In the above-described example embodiment, an exercise program is created using a model generated by the heterogeneous mixed learning. However, this is an example, and various other machine learning algorithms can be used.

(Modification 2)

The above example embodiment is directed to the system in which the evaluation report creating device 1 and the terminal devices 5 communicate with each other. Instead, the evaluation report creating device 1 may be a stand-alone device. In that case, an input device including a keyboard, a mouse, a data input connector, or the like may be provided as the input unit 21 shown in FIG. 3, and a display device or a printer may be provided as the output unit 28.

(Modification 3) The part of the user's body subjected to the creation of an exercise program and a comment may be selected and decided by specialists or users themselves. This allows the user to obtain an exercise program for training the part desired by the user in a fitness gym, for example, thereby improving the usability.

(Modification 4)

When the evaluation report is provided to the user, video contents teaching the content of the exercise program proposed in the evaluation report may also be provided to the user. The video contents provided at this time may be a real video or a video using CG.

(Modification 5)

In the above-described example embodiment, the motion extracting unit 25 extracts the motion information from the exercise video. Instead, without extracting the motion information, the exercise video itself may be inputted to the AI model as the video information.

(Modification 6)

In the above-described example embodiment, the user information is registered in the user information DB 23. However, even if the user information is not registered, the user's conditions may be evaluated from the registered exercise video and an evaluation report may be generated.

(Modification 7)

When the data of the same user is registered multiple times, the evaluation report creating device 1 may perform evaluation by comparing with the data up to the previous time, and describe the evaluation in the evaluation report.

(Modification 8)

In the above-described example embodiment, the comment creating unit 27 extracts the comment for each motion. Instead, similar past user information may be searched using all the explanatory variables contributing to the selection of each exercise, and the comments described in the evaluation report created from the user information may be used as the comments of the new evaluation report. Also, the comment creating unit 27 may extract a plurality of similar past user information from the information of the explanatory variables, and describe all the comments of the corresponding parts described in each of the evaluation reports created from them as the comments of the new evaluation report.

A part or all of the example embodiments described above may also be described as the following supplementary notes, but not limited thereto.

(Supplementary Note 1)

An information generation device comprising:

an acquisition means configured to acquire a user information relating to a user, a motion information relating to a motion of each part of a body of the user, and an evaluation report including a comment and an exercise program created by a specialist;

a storage means configured to store a model generated based on a plurality of the user information, a plurality of the motion information, and a plurality of the evaluation report;

a first creating means configured to create an exercise program for each part of one user, from the user information of the one user and the motion information of each part of the body of the one user based on the model;

a second creating means configured to create a comment indicating a basis of creating the exercise program for each part for the one user; and

a generation means configured to generate an evaluation report including the exercise program for each part and the comment indicating the basis.

(Supplementary Note 2)

The information generation device according to supplementary note 1, further comprising a motion extraction unit configured to extract the motion information from an exercise video capturing a situation in which a user is exercising.

(Supplementary Note 3)

The information generation device according to supplementary note 1 or 2, wherein the evaluation report includes a comment section indicating comments on each part of the user's body.

(Supplementary Note 4)

The information generation device according to any one of supplementary notes 1 to 3, wherein the evaluation report includes an exercise program section indicating the exercise program for each part of the user's body and a difficulty level of each exercise program.

(Supplementary Note 5)

The information generation device according to any one of supplementary notes 1 to 4, wherein the first creating means creates the exercise program for each part by heterogeneous mixed learning using the user information and the motion information as parameters.

(Supplementary Note 6)

The information generation device according to supplementary note 5, wherein the second creating means creates the basis based on the parameters and based on comments extracted from past evaluation reports.

(Supplementary Note 7)

The information generation device according to any one of supplementary notes 1 to 6, further comprising a learning means configured to make the model learn based on the user information, the motion information and the evaluation report newly acquired.

(Supplementary Note 8)

The information generation device according to any one of supplementary notes 1 to 7, further comprising an input means configured to receive a selection of the body part by the user, wherein the first creating means creates the exercise program for the part of the body selected by the user.

(Supplementary Note 9)

The information generation device according to any one of supplementary notes 1 to 8, wherein the user information includes information age, sex, a nursing degree, and a condition of body.

(Supplementary Note 10

The information generation device according to any one of supplementary notes 1 to 9, further comprising a storage unit configured to store the evaluation report including the user information, an exercise video capturing a situation in which the user is exercising, the comment created by the specialist, and the exercise program.

(Supplementary Note 11)

An information generation method comprising:

acquiring a user information relating to a user, a motion information relating to a motion of each part of a body of the user, and an evaluation report including a comment and an exercise program created by a specialist;

acquiring a model generated based on a plurality of the user information, a plurality of the motion information, and a plurality of the evaluation report;

creating an exercise program for each part of one user, from the user information of the one user and the motion information of each part of the body of the one user based on the model;

creating a comment indicating a basis of creating the exercise program for each part for the one user; and

generating an evaluation report including the exercise program for each part and the comment indicating the basis.

(Supplementary Note 12)

A recording medium recording a program causing a computer to execute a process of:

acquiring a user information relating to a user, a motion information relating to a motion of each part of a body of the user, and an evaluation report including a comment and an exercise program created by a specialist;

acquiring a model generated based on a plurality of the user information, a plurality of the motion information, and a plurality of the evaluation report;

creating an exercise program for each part of one user, from the user information of the one user and the motion information of each part of the body of the one user based on the model;

creating a comment indicating a basis of creating the exercise program for each part for the one user; and

generating an evaluation report including the exercise program for each part and the comment indicating the basis.

While the present invention has been described with reference to the example embodiments, the present invention is not limited to the above example embodiments. Various changes that can be understood by those skilled in the art within the scope of the present invention can be made in the configuration and details of the present invention. In other words, it is needless to say that the present invention includes various alterations and modifications that could be made by a person skilled in the art according to the entire disclosure, including the scope of the claims, and the technical idea. In addition, each disclosure of the above-mentioned patent references cited above shall be incorporated with reference to this document.

This application claims priority based on Japanese Patent Application 2019-171531, filed Sep. 20, 2019, and all of its disclosure is incorporated herein by reference.

### DESCRIPTION OF SYMBOLS

1 Evaluation report creating device
5 Terminal device
10, 50 Information generation device
22 Video database
23 User information database
24 Report database
25 Motion extraction unit
26 Exercise program creating unit
27 Comment creating unit

What is claimed is:

1. An information generation device comprising:
a memory configured to store instructions; and
one or more processors configured to execute the instructions to:
acquire a user information relating to a user, a motion information relating to a motion of each part of a body of the user, and an evaluation report including a comment and an exercise program created by a specialist;
store a model generated based on a plurality of the user information, a plurality of the motion information, and a plurality of the evaluation report;
create an exercise program for each part of one user, from the user information of the one user and the motion information of each part of the body of the one user based on the model;
create a comment indicating a basis of creating the exercise program for each part for the one user; and
generate an evaluation report including the exercise program for each part and the comment indicating the basis.

2. The information generation device according to claim 1, the one or more processor are further configured to extract the motion information from an exercise video capturing a situation in which a user is exercising.

3. The information generation device according to claim 1, wherein the evaluation report includes a comment section indicating comments on each part of the user's body.

4. The information generation device according to claim 1, wherein the evaluation report includes an exercise program section indicating the exercise program for each part of the user's body and a difficulty level of each exercise program.

5. The information generation device according to claim 1, wherein the one or more processors create the exercise program for each part by heterogeneous mixed learning using the user information and the motion information as parameters.

6. The information generation device according to claim 5, wherein the one or more processors create the basis based on the parameters and based on comments extracted from past evaluation reports.

7. The information generation device according to claim 1, the one or more processors are further configured to make the model learn based on the user information, the motion information and the evaluation report newly acquired.

8. The information generation device according to claim 1, the one or more processors are further configured to receive a selection of the body part by the user,
wherein the one or more processors create the exercise program for the part of the body selected by the user.

9. An information generation method comprising:
acquiring a user information relating to a user, a motion information relating to a motion of each part of a body of the user, and an evaluation report including a comment and an exercise program created by a specialist;
acquiring a model generated based on a plurality of the user information, a plurality of the motion information, and a plurality of the evaluation report;
creating an exercise program for each part of one user, from the user information of the one user and the motion information of each part of the body of the one user based on the model;
creating a comment indicating a basis of creating the exercise program for each part for the one user; and
generating an evaluation report including the exercise program for each part and the comment indicating the basis.

10. A non-transitory computer-readable recording medium recording a program causing a computer to execute a process of:
acquiring a user information relating to a user, a motion information relating to a motion of each part of a body of the user, and an evaluation report including a comment and an exercise program created by a specialist;
acquiring a model generated based on a plurality of the user information, a plurality of the motion information, and a plurality of the evaluation report;
creating an exercise program for each part of one user, from the user information of the one user and the motion information of each part of the body of the one user based on the model;
creating a comment indicating a basis of creating the exercise program for each part for the one user; and
generating an evaluation report including the exercise program for each part and the comment indicating the basis.
